(19) United States
(12) Patent Application Publication
(10) Pub. No.: US 2012/0093877 A1
(43) Pub. Date: Apr. 19, 2012

(54) COLLAGEN SCAFFOLD FOR CELL GROWTH AND A METHOD FOR PRODUCING SAME

(75) Inventor: Ming Hao Zheng, City Beach (AU)

(73) Assignee: THE UNIVERSITY OF WESTERN AUSTRALIA, Nedlands (AU)

(21) Appl. No.: 13/055,234

(22) PCT Filed: Jul. 24, 2009
(86) PCT No.: PCT/AU2009/000946

§ 371 Date: Apr. 20, 2011

(30) Foreign Application Priority Data
Jul. 24, 2008 (AU) 2008903789

(51) Int. Cl. A61K 9/00; A61P 41/00; D04H 13/00; C07K 1/107

(52) U.S. Cl. 424/400; 530/410; 442/304

(57) ABSTRACT

A bioscaffold and method of manufacture is described. The bioscaffold comprised greater than 80% type I collagen fibers or bundles having a knitted structure providing tensile load strength. The method of manufacture comprises the steps of: (a) isolating collagen fibers or bundles; (b) incubating said fibers or bundles in a mixture of NaOH, alcohol, acetone, HC; and ascorbic acid; and (c) mechanical manipulation of said fibers or bundles to produce a knitted structure.

### FIELD OF THE INVENTION

The present invention relates to bioscaffolds and methods of manufacturing bioscaffolds. In particular the invention relates to a bioscaffold comprising greater than 80% type I collagen fibers or bundles having a knitted structure providing mechanical strength and elasticity.

### INTRODUCTION

Bioscaffolds are structures that replace an organ or tissue temporarily or permanently to aid the restoration of normal function. The bioscaffold provides a substrate on which cells proliferate and differentiate, eventually replacing the bioscaffold and restoring normal organ or tissue function.

There are a number of properties that are desirable in a bioscaffold, these are: a) interconnecting pores that favour tissue integration and vascularisation; b) appropriately biodegrade and bioresorb such that de novo tissue ultimately replaces the scaffold; c) surface chemistry that promotes cell attachment, proliferation and differentiation; d) adequate mechanical properties; e) does not induce adverse biological responses; and f) easily fabricated in a variety of shapes and sizes.

In pursuit of bioscaffolds with the properties listed supra, tissue engineers have fabricated scaffolds from both synthetic and naturally derived materials. For example, bioscaffolds have been made from synthetic polymers such as polyglycolic acid, polylactic acid and their copolymers. Naturally derived materials from which bioscaffolds are made include protein and carbohydrate polymers. As well as being fabricated from various materials, bioscaffolds have been manufactured in different forms such as membranes, microbeads, fleece, fibers and gels.

However, currently available bioscaffolds have a number of drawbacks. Synthetic polymer scaffolds do not possess surface chemistry familiar to cells and therefore cell attachment is suboptimal. Further, synthetic polymer scaffolds produce acidic by-products when degraded which reduces the local pH and disrupts the cell microenvironment, discouraging normal cell growth.

Currently available bioscaffolds fabricated from naturally derived materials also have a number of disadvantages. These bioscaffolds often elicit immune responses due to presence of residual foreign cells from the host from which the material was isolated. Further, the pore size and structure of these scaffolds generally does not optimally promote cell growth and tissue vascularisation. Lastly, the bioscaffolds currently available lack sufficient mechanical properties required to withstand the harsh environments in which bioscaffolds are regularly used, for example joint repair.

Accordingly, there is a need to develop a bioscaffold that better promotes cell growth and has improved mechanical properties.

### SUMMARY OF THE INVENTION

The inventors of the present invention have developed a method for producing a novel bioscaffold comprising collagen fibers or bundles which have improved properties including superior mechanical strength compared to currently available collagen bioscaffolds.

Accordingly, in a first aspect the present invention provides a bioscaffold comprising greater than 80% type I collagen fibers or bundles having a knitted structure and a maximum tensile load strength of greater than 20N.

In some embodiments, the maximum tensile load strength of the bioscaffold is greater than 40N. In other embodiments, the maximum tensile load strength is greater than 60N. In another embodiment, the maximum tensile load strength is greater than 120N. In still other embodiments, the maximum tensile load strength is greater than 140N.

In some embodiments, the bioscaffold has a modulus of greater than 100 MPa. In other embodiments, the modulus is greater than 200 MPa. In another embodiment, the modulus is greater than 300 MPa. In still other embodiments, the modulus is greater than 400 MPa. In still further embodiments, the modulus is greater than 500 MPa.

In some embodiments, the bioscaffold has an extension at maximum load of less than 85%. In other embodiments, the extension at maximum load is less than 80%.

In some embodiments, the bioscaffold comprises greater than 85% type I collagen. In other embodiments, the bioscaffold comprises greater than 90% type I collagen.

In some embodiments, the bioscaffold has a knitted structure comprising first and second groups of collagen fibers or bundles where fibers or bundles in the first group extend predominately in a first direction and fibers or bundles in the second group extend predominately in a second direction.

In some embodiments, the first and second directions of the groups of collagen fibers or bundles are substantially perpendicular to each other. In other embodiments, the fibers or bundles in the first group are generally spaced apart from each other by a first distance and the fibers or bundles in the second group are generally spaced apart from each other by a second distance and where the first and second distances are different to each other. In still further embodiments, the different fibers or bundles of the first group overly, or underlie or weave through fibers or bundles of the second group.

In a second aspect, the present invention provides a bioscaffold comprising greater than 80% type I collagen fibers or bundles having a knitted structure and has an extension at maximum load of less than 85%.

In a third aspect, the present invention provides a bioscaffold comprising greater than 80% type I collagen fibers or bundles having a knitted structure and a maximum tensile load strength of greater than 20N, a modulus of greater than 100 MPa and an extension at maximum load of less than 85%.

In a fourth aspect, the present invention provides a method of manufacturing a bioscaffold comprising the steps of: (a) isolating collagen fibers or bundles from a mammal; (b) incubating said fibers or bundles in a mixture of NaOH, alcohol, acetone, HCl and ascorbic acid; and (c) mechanical manipulation of said fibers or bundles to produce a knitted structure.

In some embodiments, the collagen fibers or bundles of the bioscaffold are provided from dense connective tissue. It will be appreciated that the dense connective tissue used in this embodiment of the bioscaffold as described herein can be isolated from any tissue containing dense connective tissue. In some embodiments the tissue is a tendon. In other embodiments the tissue is epitendon. The tendon or epitendon may be from any tendon from any anatomical site of an animal and may be a rotator cuff tendon, supraspinatus tendon, subcapularis tendon, pectroalis major tendon, peroneal tendon, achille's tendon, tibialis anterior tendon, anterior cruciate ligament, posterior cruciate ligament, hamstring tendon, lateral ligament, medial ligament, patella tendon, biceps tendon, and triceps tendon.

In some embodiments, the dense connective tissue may be isolated from any mammalian animal including, but not limited to a sheep, a cow, a pig or a human. In other embodiments, the dense connective tissue is isolated from a human. In still other embodiments the dense connective tissue is autologous.

The present invention also provides a method of repairing a tissue defect in a mammalian animal comprising implanting at the site of the tissue defect a bioscaffold according to the an embodiment of the present invention.

Accordingly, in a fifth aspect the present invention provides a method of repairing a tissue defect in a mammalian animal comprising implanting at the site of the tissue defect a bioscaffold comprising greater than 80% type I collagen fibers or bundles having a knitted structure and a maximum tensile load strength of greater than 20N, a modulus of greater than 100 MPa and an extension at maximum load of less than 85%.

In some embodiments, the mammalian animal is a human.

### BRIEF DESCRIPTION OF THE FIGURES

FIG. 1: Confocal image (×20) of a bioscaffold in accordance with an embodiment of the invention.

FIG. 2: Scanning electron microscopy (SEM) image (×100) of a bioscaffold shown in FIG. 1.

FIG. 3: Scanning electron microscopy (SEM) image (×1000) of the bioscaffold of the invention.

FIG. 4: Confocal image of a commercially available bioscaffold (SIS/Lycol collagen membrane).

FIG. 5: Scanning electron microscopy (SEM) image (×200) of a commercially available bioscaffold (“Bio-gide”).

FIG. 6: Scanning electron microscopy (SEM) image (×1500) of a commercially available bioscaffold (Lycol collagen membrane).

FIG. 7: Is a graph showing comparative load-extension curves for a bioscaffold in accordance with an embodiment of the invention and another commercially available collagen membrane.

FIG. 8: Is a bar graph showing comparative mean modulus for bioscaffolds in accordance with the present invention and commercially available Bio-gide collagen membrane scaffolds;

FIG. 9: Is a bar graph showing comparative mean maximum load for bioscaffolds in accordance with the present invention and commercially available Bio-gide collagen membrane scaffolds;

FIG. 10: Is a bar graph showing comparative mean extension at maximum load for bioscaffolds in accordance with the present invention and commercially available Bio-gide collagen membrane scaffolds;

FIG. 11: Is a bar graph showing comparative mean load at yield for bioscaffolds in accordance with the present invention and commercially available Bio-gide collagen membrane scaffolds; and,

FIG. 12: Is a bar graph showing comparative mean extension at yield for bioscaffolds in accordance with the present invention and commercially available Bio-gide collagen membrane scaffolds.

FIG. 13: Is a light micrograph comparing loose connective tissue (LCT) and dense connective tissue (DCT) from the mammary gland stained with haematoxylin and eosin (from Kastelic et al. “The Multicomposite structure of Tendon” Connective Tissue Research, 1978, Vol. 6, pp. 11-23). Epithelium (EP) is also shown.

FIG. 14: Is a schematic diagram of the tendon (adapted from Kastelic et al. “The Multicomposite structure of Tendon” Connective Tissue Research, 1978, Vol. 6, pp. 11-23).

### DETAILED DESCRIPTION OF THE PREFERRED EMBODIMENTS

Before describing embodiments of the present invention in detail, it is to be understood that this invention is not limited to particularly exemplified methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell culture, cell biology and tissue engineering, which are within the skill of the art. Such techniques are described in the literature. See, for example, Coligan et al., 1999 “Current protocols in Protein Science” Volume I and II (John Wiley & Sons Inc.); Ross et al., 1995 “Histology: Text and Atlas”, 3^{rd} Ed., (Williams & Wilkins); Kruse & Patterson (eds.) 1977 “Tissue Culture” (Academic Press); and Alberts et al. 2000 “Molecular Biology of the Cell” (Garland Science).

It must be noted that as used herein and in the appended claims, the singular forms “a”, “an”, and “the” include plural reference unless the context clearly dictates otherwise. Thus, for example, a reference to “a cell” includes a plurality of such cells, and a reference to “an agent” is a reference to one or more agents, and so forth. Unless defined otherwise, all technical and scientific terms used herein have the same meanings as commonly understood by one of ordinary skill in the art to which this invention belongs.

Although any materials and methods similar or equivalent to those described herein can be used to practice or test the present invention, the preferred materials and methods are now described.

In some embodiments the present invention is directed towards a bioscaffold comprising collagen fibers or bundles.

Collagen bundles are composed of collagen fibers. Collagen fibers are composed of three polypeptide chains that intertwine to form a right-handed triple helix. Each collagen polypeptide chain is designated as an α chain and is rich in glycine, proline and hydroxyproline. There are a number of different α chains and different combinations of these α chains correspond with different types of collagen. In some embodiments, the bioscaffold of the present invention comprises type I collagen. Type I collagen is composed of two α1 chains and one α2 chain.

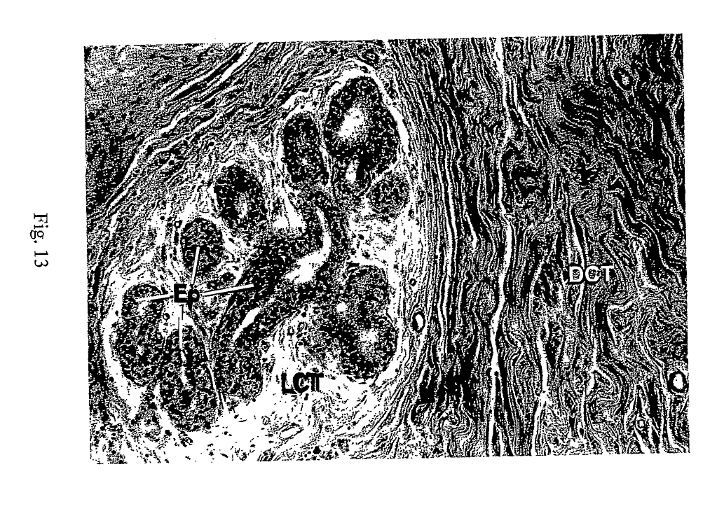

In some embodiments, the collagen fibers or bundles are provided from dense connective tissue isolated from a source. The term “dense connective tissue” as used herein refers to the matrix comprised primarily of type I collagen fibers or bundles found in the tendons, ligaments and dermis of all mammals. As illustrated in FIG. 13, dense connective tissue is distinct from “loose connective tissue”. Loose connective tissue is characterised by loosely arranged fibers and an abundance of cells and is present, for example, beneath the epithelia that covers body surfaces and lines internal organs.

Dense connective tissue may be regular or irregular. Dense regular connective tissue provides strong connection between different tissues and is found in tendons and ligaments. The collagen fibers in dense regular connective tissue are bundled in a parallel fashion. Dense irregular connective tissue has fibers that are not arranged in parallel bundles as in dense regular connective tissue and comprises a large portion of the dermal layer of skin. The bioscaffold of the present invention may be composed of either regular dense connective tissue or dense irregular connective tissue, or a combination of both.

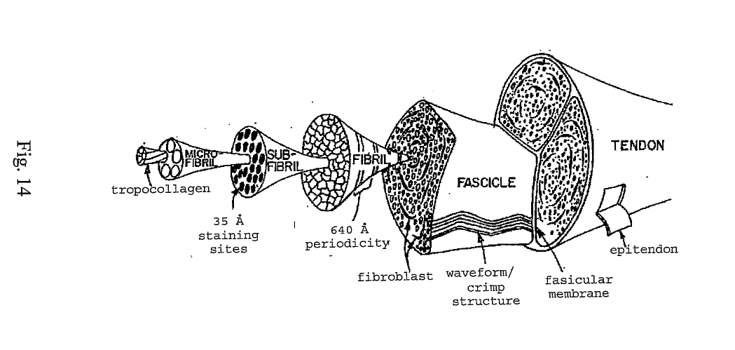

The term “source” as used herein refers to any tissue containing dense connective tissue in any mammal. In some embodiments, the tissue containing dense connective tissue is a tendon. A tendon is the tissue which connects muscle to bone in a mammal. In other embodiments the tissue is epitendon. Epitendon is the thin connective tissue capsule that surrounds the substance of the tendon, as illustrated in FIG. 14.

The tendon may be from any anatomical site of an mammal and may be a rotator cuff tendon, supraspinatus tendon, subcapularis tendon, pectroalis major tendon, peroneal tendon, achille's tendon, ibialis anterior tendon, anterior cruciate ligament, posterior cruciate ligament, hamstring tendon, lateral ligament, medial ligament, patella tendon, biceps tendon, and triceps tendon. The epitendon may also be isolated from any of the above tendons.

Tendon may be isolated from a source in a variety of ways, all which are known to one skilled in the art. In some embodiments, a section of tendon can be isolated by biopsy using conventional methods.

In some embodiments, the tissue containing dense connective tissue may be isolated from any mammalian animal including, but not limited to a sheep, a cow, a pig or a human. In other embodiments, the tissue containing dense connective tissue is isolated from a human.

In some embodiments, the tissue containing dense connective tissue is “autologous”, i.e. isolated from the body of the subject in need of treatment. For example, a mammalian subject with a rotator cuff tear can have a biopsy taken from any tendon in their body. Such tendons include, but are not limited to, tendon of flexor carpi radialis and the calcaneus tendon.

In some embodiments, the present invention provides a bioscaffold comprising greater than 80% type I collagen. In other embodiments, the bioscaffold comprises at least 85% type I collagen. In still other embodiments the bioscaffold comprises greater than 90% type I collagen.

The collagen fibers or bundles of the bioscaffold form a knitted structure. The term “knitted structure” as used herein refers to a structure comprising first and second groups of fibers or bundles where fibers or bundles in the first group extend predominately in a first direction and fibers or bundles in the second group extend predominately in a second direction, where the first and second directions are different to each other and the fibers or bundles in the first group interleave or otherwise weave with the fibers or bundles in the second group. The difference in direction may be about 90°.

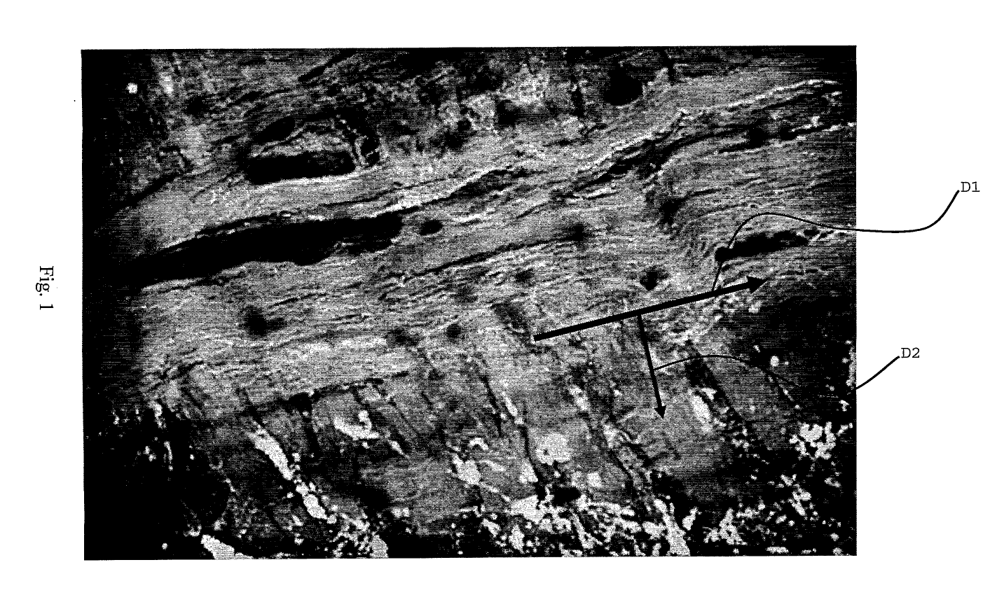

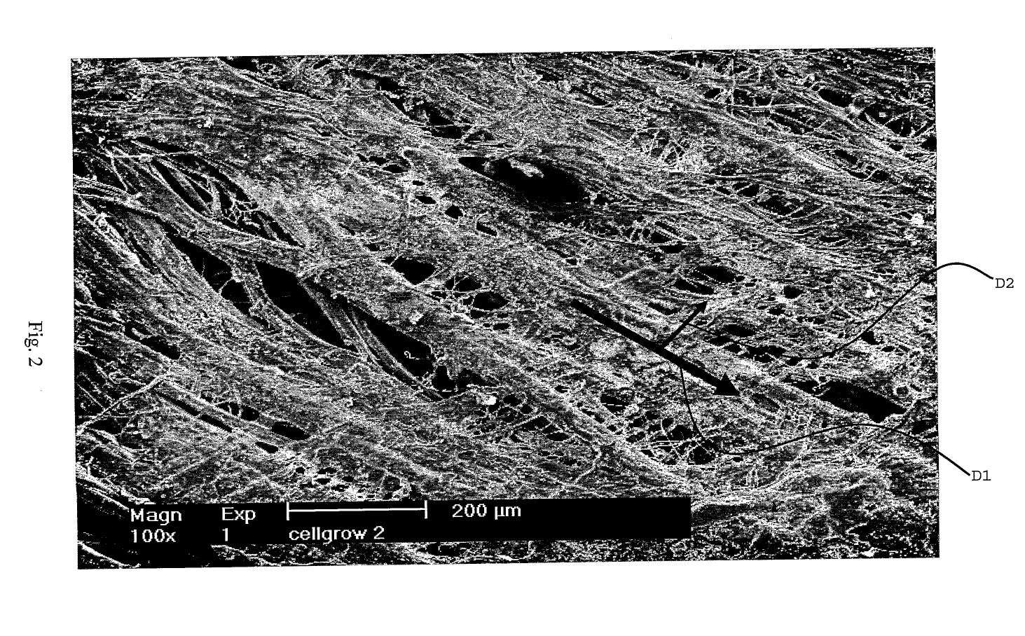

FIGS. 1-3 depict the physical structure of an embodiment of the bioscaffold at increasing magnifications of 20, 100 and 1000 times respectively. As is evident from these Figures, embodiments of the bioscaffold are characterised by a knitted structure of fibers or bundles. This knitted structure applies to both collagen fibers or bundles and elastin fibers. The knitted structure comprises a first group of fibers or bundles extending in a first direction D1 and a second group of fibers or bundles extending in a second direction D2 that is different to, and indeed in this embodiment at approximately 90° to direction D1. The fibers or bundles in each group interweave with each other forming a porous structure promoting cell growth within the bioscaffold.

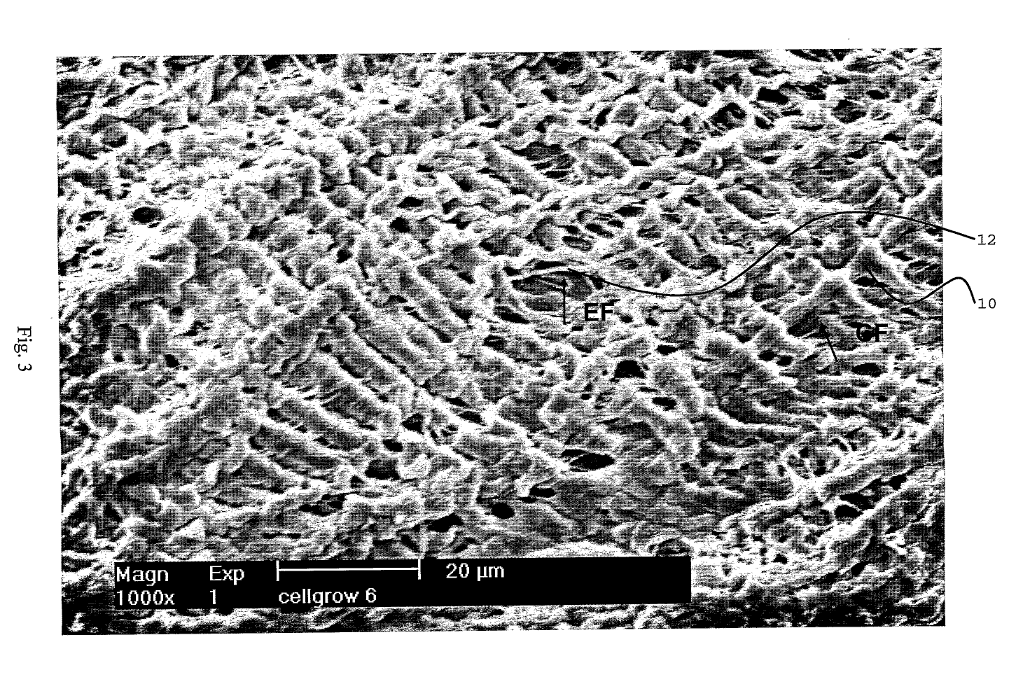

FIG. 3 depicts both collagen fibers or bundles 10 and elastin fibers 12. The collagen fibers or bundles 10 are differentiated from the elastin fibers 12 by their greater thickness and twisted configuration.

As is further apparent from FIG. 3, a present embodiment of the bioscaffold is composed largely of collagen fibers or bundles 10. In particular, the collagen fibers or bundles 10 may be provided in an amount of approximately 80%-90% of type 1 collagen fibers or bundles with the elastin fibers 12 being provided in an amount of between 10-20%. The remaining portion of the fibre content of the bioscaffold is provided by other types of collagen fibers or bundles including type III, type IV, type V and type X.

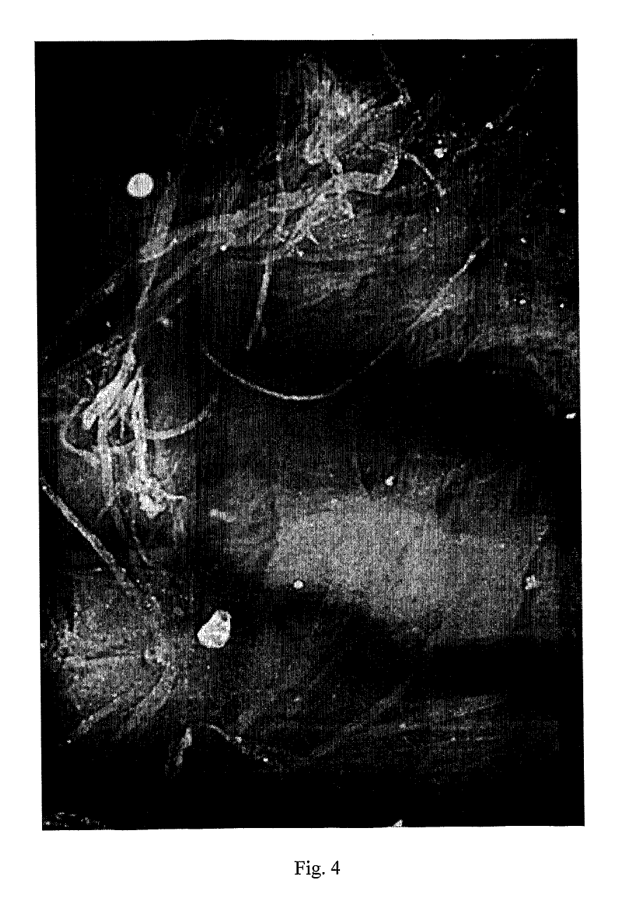

It is believed that the knitted structure of embodiments of the present bioscaffold provide superior mechanical properties to those of currently known bioscaffolds. The difference in structure is exemplified by consideration of currently available bioscaffolds depicted in FIGS. 4-6.

FIG. 4 is a confocal image of commercially available SIS/Lycol collagen membranes. This clearly depicts a random arrangement of collagen bundles and fibers.

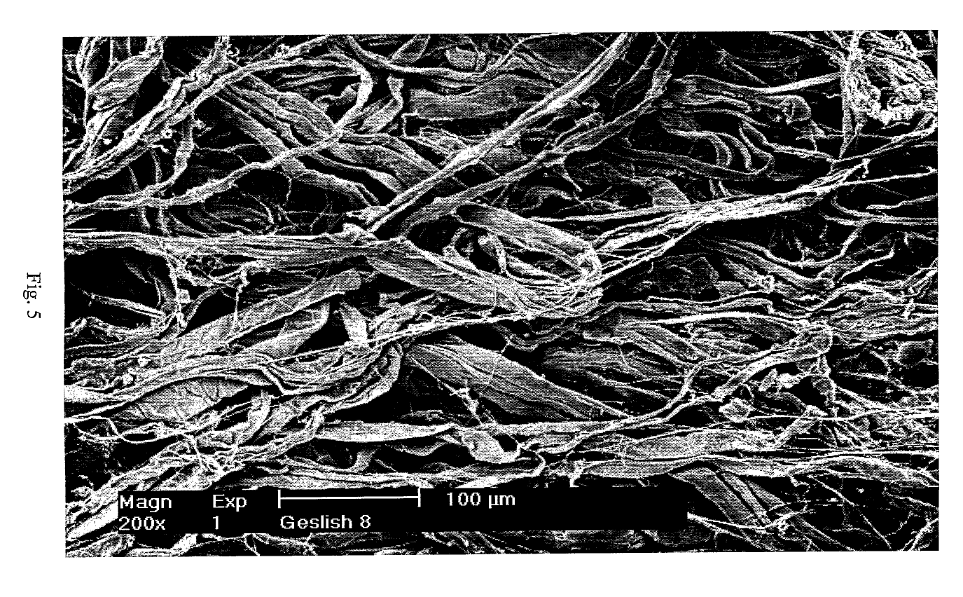

FIG. 5 provides a scanning electron microscope image at 200 times magnification of the commercially available bio-gide collagen membrane. The random arrangement of collagen and elastin fibers is clearly evident and readily distinguishable from the knitted structure shown in FIG. 3.

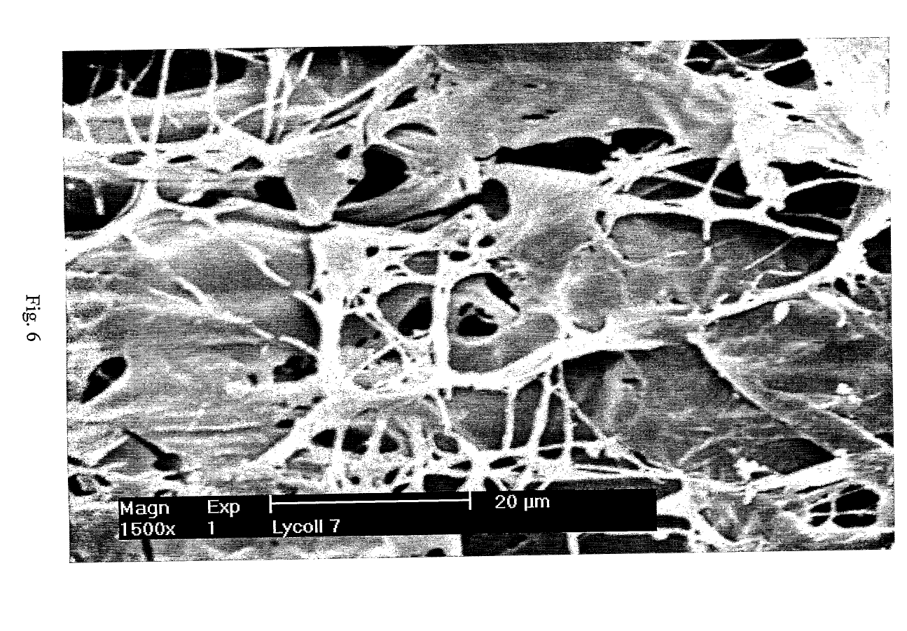

FIG. 6 is a scanning electron microscope image at 1500 times magnification of a commercially available Lycol collagen membrane. This clearly displays a random distribution of collagen fibers in a collagen “gel” matrix.

It is believed that the knitted structure in embodiments of the present invention provide increased maximum tensile load strength compared to currently available scaffolds.

The term “maximum tensile load strength” as used herein refers to the maximum tensile load that the bioscaffold can bear. On a Load v Extension curve this is represented by the peak load on the curve.

In some embodiments, the bioscaffold has maximum tensile load strength of greater than 20N. In some embodiments, the bioscaffold of the present invention has maximum tensile load strength greater than 25N, 40N, 60N, 80N, 100N, 120N or 140N.

Further, it is believed that the knitted structure of the embodiments of the bioscaffold provides reduced extension at maximum load of the bioscaffold while providing an increase in modulus.

The term “modulus” as used herein means Young's Modulus and is determined as the ratio between stress and strain. This provides a measure of the stiffness of the bioscaffold.

In some embodiments the bioscaffold has a modulus of greater than 100 MPa. In other embodiments the bioscaffold has a modulus of greater than 200 MPa, 300MPa, 400 MPa, or 500 MPa.

The term “extension at maximum load” as used herein means the extension of the bioscaffold at the maximum tensile load strength referenced to the original length of the bioscaffold in a non-loaded condition. This is to be contrast with maximum extension which will be greater.

In some embodiments, the bioscaffold has extension at maximum load of less than 85% of the original length.

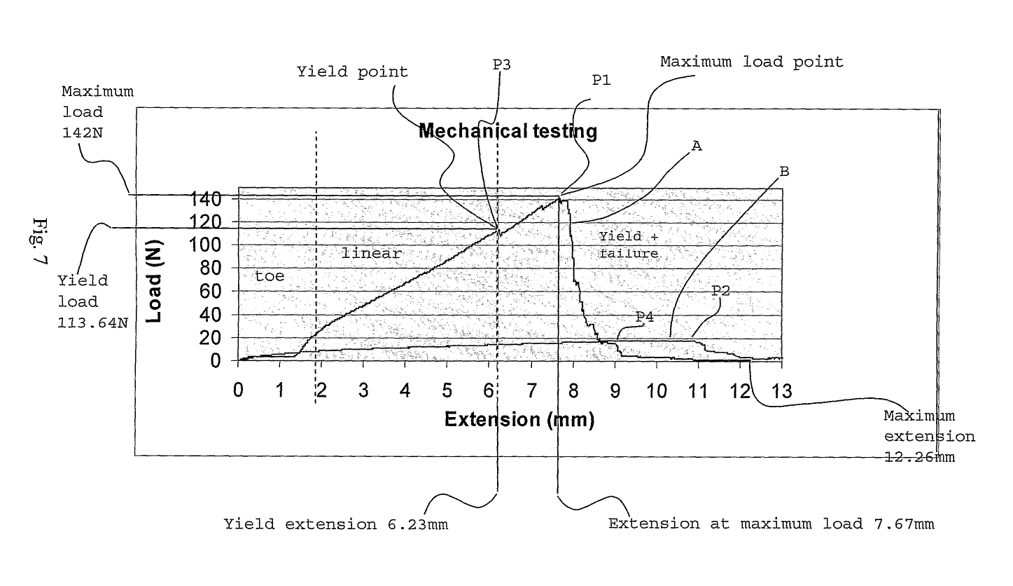

FIG. 7 depicts a comparison of the Load v Extension curve of a bioscaffold in accordance with an embodiment of the present invention, depicted as curve A; and, a currently available bio-gide collagen membrane scaffold, depicted as curve B. The initial length of both scaffolds tested is 10 mm. Accordingly, in this particular test, where the extension is also shown in millimetres, the extension in millimetres corresponds with a percentage increase in extension. For example, an extension of 6 mm represents an extension of 60% of the at rest unloaded scaffold.

It is noted that curve A has a shape that approximates the upwardly concave shape of the Load v Extension curve for a tendon or ligament in that it includes a toe region, a linear region and a yield and failure region. In a tendon or ligament, the toe region is characterised by crimps being removed by elongation. The linear region is characterised by molecular cross-links of collagen being stressed. This region is indicative of the stiffness of the tendon or ligament. The yield and failure region is characterised by the onset of cross-link or fibre damage leading ultimately to failure.

Point P1 on curve A in FIG. 7 shows a maximum tensile load strength of 140.63N of the tested embodiment of the bioscaffold. The extension of the bioscaffold at this maximum load is 7.67 mm. As the initial at rest length of the tested bioscaffold is 10 mm, this represents an extension of 76.7%. In contrast, the maximum tensile load strength P2 of the prior art scaffold shown in curve B is approximately 19N and provides an extension of approximately 10.9 mm equating to a 100.9% extension in length.

Point P3 on the curve A shown in FIG. 7 represents the yield point of the present tested embodiment of the bioscaffold. The yield point is the point at which the bioscaffold commences to fail. Beyond the yield point, upon relaxation of the tensile load, the scaffold will not return to its original length. It remains plastically deformed. The yield point for the tested embodiment of the bioscaffold is at a tensile load of approximately 114N and provides an extension of approximately 6.25 mm representing a 62.5% increase in length. With the prior art scaffold shown by curve B, the yield point is difficult to discern but may be approximated by point P4 on curve B at a load of approximately 19.4N giving an extension of approximately 9 mm or 90%.

In order to maximise the mechanical force transmission efficiency of a tendon, it is desirable for the tendon to undergo low extension under physiological conditions. It is therefore believed to be beneficial for bioscaffolds used in tendon and ligament repair to have minimal extension at maximum extension. This property may also be gauged by assessing the modulus of the bioscaffold. As the modulus is a measure of the stiffness, it is desirable to have a relatively high modulus.

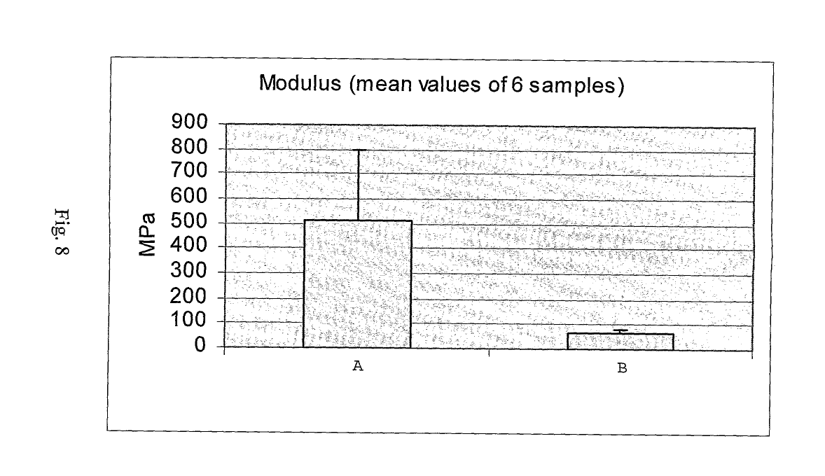

FIG. 8 graphically represents the mean modulus of six samples of: an embodiment of the bioscaffold in accordance with the present invention, depicted as bar A, and the prior art Bio-gide collagen membrane, depicted by bar B.

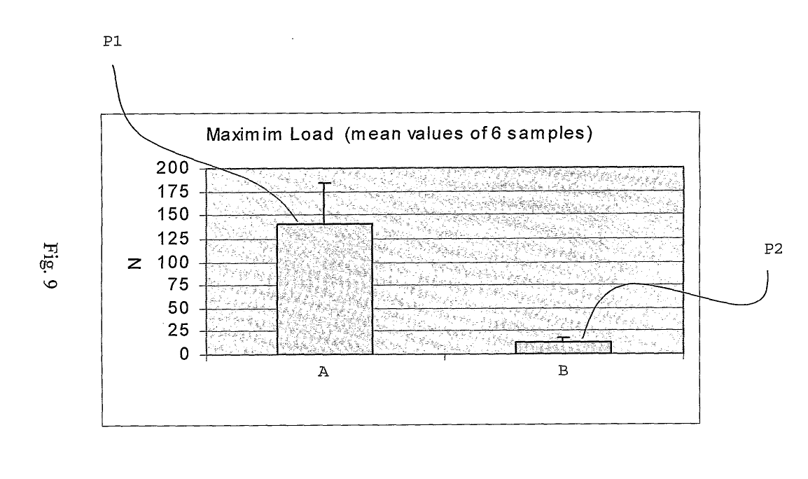

FIG. 9 graphically depicts a comparison of the mean maximum load (ie, mean maximum tensile load strength) of embodiments of the present bioscaffold shown as bar A, and the prior art scaffold shown as bar B. The upper horizontal line on bar A is commensurate with point P1 on curve A shown in FIG. 7. The upper horizontal bar on bar B in FIG. 9 is representative of the point P2 on curve B in FIG. 7.

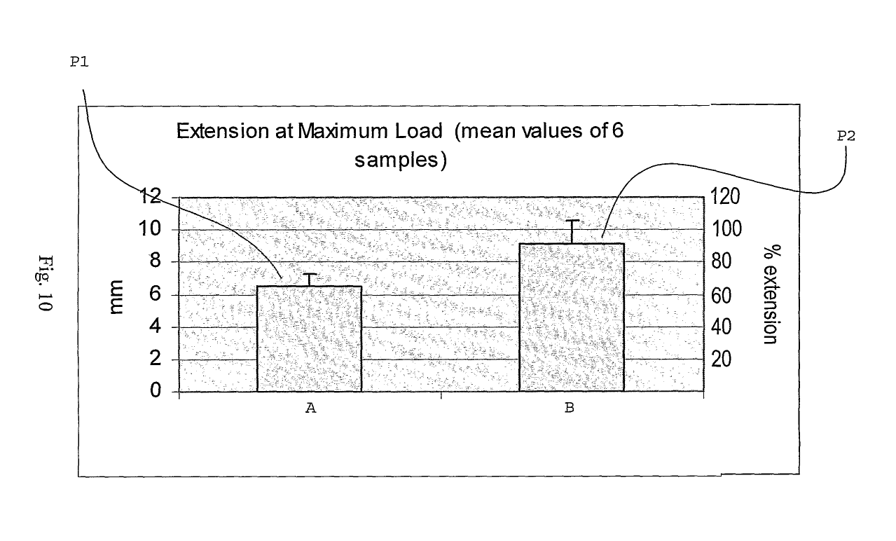

FIG. 10 graphically depicts the mean extension at maximum load of embodiments of the present scaffold, depicted by bar A, and of the prior art scaffold, depicted by bar B. The upper horizontal line on bar A in FIG. 10 is commensurate with the extension shown in FIG. 7 at the point P1 on curve A. Similarly, the horizontal bar P2 on bar B in FIG. 10 is commensurate with the extension at point P2 on curve B in FIG. 7.

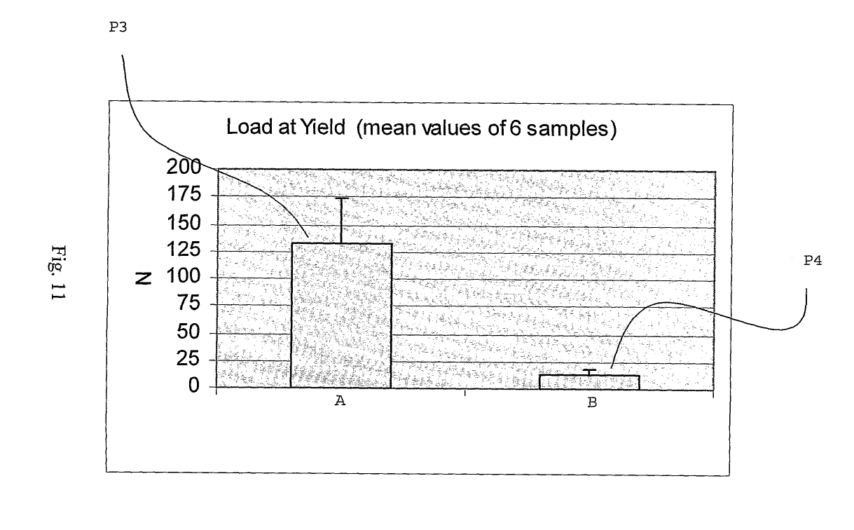

FIG. 11 depicts the mean yield point (ie, tensile load at yield) for embodiments of the present scaffold, depicted by bar A: and, for the prior art, depicted by bar B. The upper horizontal line P3 on bar A of FIG. 11 is commensurate with the load at point P3 on curve A in FIG. 7. Similarly, the upper horizontal bar P4 on bar B in FIG. 11 is commensurate with the load at point P4 shown in curve B on FIG. 7.

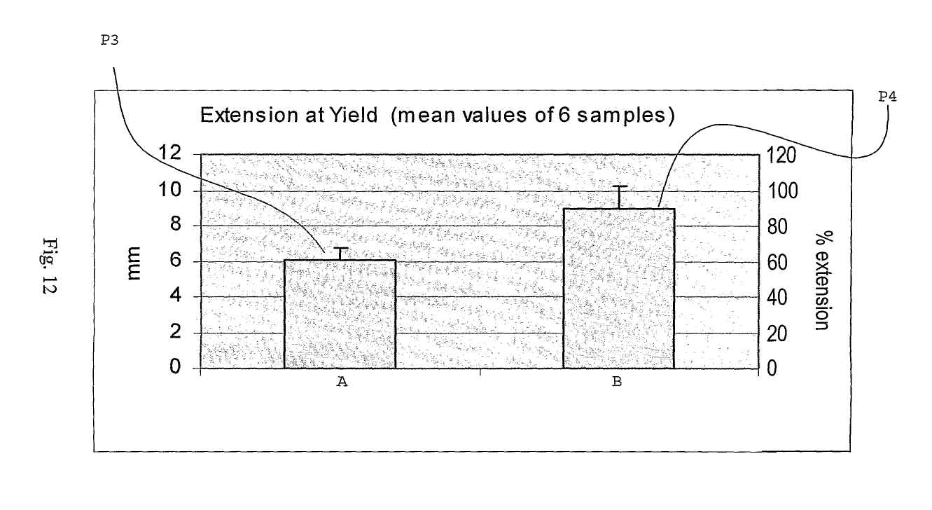

FIG. 12 depicts the mean extension at yield of embodiments of the present scaffold in bar A, and for the prior art scaffold in bar B. The upper horizontal line P3 on bar A in FIG. 12 is commensurate with the extension at point P3 on curve A in FIG. 7, while the upper horizontal bar on bar B in FIG. 12 is commensurate with the extension at point P4 on curve B in FIG. 7.

Naturally, aspects of the present invention also encompass methods of manufacturing the scaffold described in detail above. As previously discussed, the bioscaffold is composed of dense connective tissue. Accordingly, the first step in manufacturing the scaffold comprises isolating collagen fibers or bundles from a mammal. Sources of collagen fibers or bundles would be known to a person skilled in the art and are also discussed supra.

The collagen fibers or bundles once isolated are incubated in a solution of NaOH, alcohol, acetone, HCl and ascorbic acid in a warm and cold cycle and under vacuum conditions. The fibers or bundles are then mechanically manipulated in order to flatten the surface of the scaffold and produce a knitted structure described above.

The bioscaffold of the present invention may be used in repairing a tissue defect in a mammalian animal. It will be appreciated that the tissue in need of repair may be any tissue found in a mammalian animal, including but not limited to epithelium, connective tissue or muscle.

The terms “repairing” or “repair” or grammatical equivalents thereof are used herein to cover the repair of a tissue defect in a mammalian animal, preferably a human. “Repair” refers to the formation of new tissue sufficient to at least partially fill a void or structural discontinuity at a tissue defect site. Repair does not however, mean or otherwise necessitate, a process of complete healing or a treatment, which is 100% effective at restoring a tissue defect to its pre-defect physiological/structural/mechanical state.

The term “tissue defect” or “tissue defect site”, refers to a disruption of epithelium, connective or muscle tissue. A tissue defect results in a tissue performing at a suboptimal level or being in a suboptimal condition. For example, a tissue defect may be a partial thickness or full thickness tear in a tendon or the result of local cell death due to an infarct in heart muscle. A tissue defect can assume the configuration of a “void”, which is understood to mean a three-dimensional defect such as, for example, a gap, cavity, hole or other substantial disruption in the structural integrity of the epithelium, connective or muscle tissue. In certain embodiments, the tissue defect is such that it is incapable of endogenous or spontaneous repair. A tissue defect can be the result of accident, disease, and/or surgical manipulation. For example, cartilage defects may be the result of trauma to a joint such as a displacement of torn meniscus tissue into the joint. Tissue defects may be also be the result of degenerative diseases such as osteoarthritis.

Typically, the bioscaffold of the invention will be implanted at the site of the tissue defect and secured in place by any conventional means known to those skilled in the art, e.g. suturing, suture anchors, bone fixation devices and bone or biodegradable polymer screws.

1. A bioscaffold comprising greater than 80% type I collagen fibers or bundles having a knitted structure and a maximum tensile load strength of greater than 20N.

2. The bioscaffold of claim 1, wherein the maximum tensile load strength is greater than 40N.

3. The bioscaffold of claim 1, wherein the maximum tensile load strength is greater than 60N.

4. The bioscaffold of claim 1, wherein the maximum tensile load strength is greater than 80N.

5. The bioscaffold of claim 1, wherein the maximum tensile load strength is greater than 100N.

6. The bioscaffold of claim 1, wherein the maximum tensile load strength is greater than 120N.

7. The bioscaffold of claim 1, wherein the maximum tensile load strength is greater than 140N.

8. The bioscaffold of claim 1, wherein the bioscaffold has a modulus of greater than 100 MPa.

9. The bioscaffold of claim 8, wherein the bioscaffold has a modulus of greater than 200 MPa.

10. The bioscaffold of claim 9, wherein the bioscaffold has a modulus of greater than 300 MPa.

11. The bioscaffold of claim 10, wherein the bioscaffold has a modulus of greater than 400 MPa.

12. The bioscaffold of claim 11, wherein the bioscaffold has a modulus of greater than 500 MPa.

13. The bioscaffold of claim 1, wherein the bioscaffold has an extension at maximum load of less than 85%.

14. The bioscaffold of claim 13, wherein the bioscaffold has an extension at maximum load of less than 80%.

15. The bioscaffold of claim 1, comprising greater than 85% type I collagen.

16. The bioscaffold of claim 1, comprising greater than 90% type I collagen.

17. A bioscaffold comprising greater than 80% type I collagen fibers or bundles having a knitted structure and a maximum tensile load strength of greater than 20N, a modulus of greater than 100 MPa and an extension at maximum load of less than 85%.

18. A bioscaffold comprising greater than 80% type I collagen fibers or bundles having a knitted structure and has an extension at maximum load of less than 85%.

19. A bioscaffold comprising greater than 80% type I collagen fibers or bundles having a knitted structure and has an extension at maximum load of less than 80%.

20. The of claim 1, wherein the knitted structure comprises first and second groups of collagen fibers or bundles where fibers or bundles in the first group extend predominately in a first direction and fibers or bundles in the second group extend predominately in a second direction.

21. The bioscaffold of claim 20, wherein the first and second directions are substantially perpendicular to each other.

22. The bioscaffold of claim 20, where the fibers or bundles in the first group are generally spaced apart from each other by a first distance and the fibers or bundles in the second group are generally spaced apart from each other by a second distance and where the first and second distances are different to each other.

23. The bioscaffold of claim 20, and wherein different fibers or bundles of the first group overly, or underlie or weave through the fibers or bundles of the second group.

24. A method of manufacturing a bioscaffold comprising:
(a) isolating collagen fibers or bundles from a mammal;
(b) incubating said fibers or bundles in a mixture of NaOH, alcohol, acetone, HCl and ascorbic acid; and
(c) mechanical manipulation of said fibers or bundles to produce a knitted structure.

25. The method of claim 24, wherein the collagen fibers or bundles are provided from dense connective tissue.

26. The method of claim 25, wherein the dense connective tissue is isolated from a sheep, a cow, a pig or a human.

27. The method of claim 25, wherein the dense connective tissue is isolated from a human.

28. The method of claim 25, wherein the dense connective tissue is autologous.

29. The method of claim 25, wherein the dense connective tissue is isolated from a tendon.

30. The method according to any one of claim 25, wherein the dense connective tissue is isolated from the epitendon of a tendon.

31. The method of claim 29, wherein the tendon is selected from the group consisting of a rotator cuff tendon, a supraspinatus tendon, a subcapularis tendon, a pectroalis major tendon, a peroneal tendon, a achille's tendon, a tibialis anterior tendon, a anterior cruciate ligament, a posterior cruciate ligament, a hamstring tendon, a lateral ligament, a medial ligament, a patella tendon, a biceps tendon, and a triceps tendon.

32. A method of repairing a tissue defect in a mammalian animal comprising implanting at the site of the tissue defect the bioscaffold of claim 1.

33. A method of repairing a tissue defect in a mammalian animal comprising implanting at the site of the tissue defect a bioscaffold comprising greater than 80% type I collagen fibers or bundles having a knitted structure and a maximum tensile load strength of greater than 20N, a modulus of greater than 100 MPa and an extension at maximum load of less than 85%.

34. The method of claim 33, wherein the mammalian animal is a human.
